(19) United States
(12) Patent Application Publication
(10) Pub. No.: US 2017/0258506 A1
(43) Pub. Date: Sep. 14, 2017

(54) Injection Devices For Joint Fixation And Related Methods

(71) Applicant: West End Bay Partners, LLC, Tyler, TX (US)

(72) Inventors: Andy J. Redmond, Tyler, TX (US); Charles R. Gordon, Tyler, TX (US); Erik Wagner, Austin, TX (US)

(21) Appl. No.: 15/453,762

(22) Filed: Mar. 8, 2017

Related U.S. Application Data
(60) Provisional application No. 62/305,406, filed on Mar. 8, 2016.

(51) Int. Cl. A61B 17/88; A61B 17/86

(52) U.S. Cl. CPC A61B 17/8816; A61B 17/8819; A61B 17/8811; A61B 17/8822; A61B 17/8808; A61B 17/863; A61B 2017/564

(57) ABSTRACT

An injection device for use in sacroiliac joint stabilization comprises an elongated conduit having a first end and a second end, where the first end includes a first opening and the second end includes a second opening; a carrier member couplable with the conduit and configured to accommodate a guidewire; a tool configured to be at least partially disposed in a bore of the conduit via the second opening, where the tool is configured to urge a hardening material in the bore toward the first end of the conduit; where the first opening of the conduit is configured to be angularly disposed relative to the second opening of the conduit such that the first end directs hardening material in a non-parallel direction relative to a longitudinal axis of the conduit.

### CROSS-REFERENCE TO RELATED APPLICATIONS

This application claims priority to U.S. Provisional Application No. 62/305,406, filed on Mar. 8, 2016, the entire contents of which is specifically incorporated by reference herein without disclaimer.

### BACKGROUND OF THE INVENTION

1. Field of the Invention

The present invention relates generally to injection devices for joint fixation, and more particularly, but not by way of limitation, to injection devices for use in medical procedures in which a hardening material is injected into one or more cavities on opposing sides of a joint.

2. Description of Related Art

The sacroiliac joint is a strong, weight-bearing synovial joint and part of the pelvis. The sacroiliac joint comprises strong ligamentous attachments and two articular dense cortical surfaces: the sacrum and the ilium. The body of each of the sacrum and the ilium includes soft cancellous bone.

The sacroiliac joint has been identified as a source of pain and disability in a large number of patients. Pain of the sacroiliac joint may be a result of bone degeneration, fracture, dislocation, and/or trauma. Current treatment management options include sacroiliac joint fusion. Traditional open surgical techniques for sacroiliac joint fusion carry a significant risk for complications. Percutaneous sacroiliac joint fusion, however, has emerged as a minimally invasive option for treating sacroiliac joint pain. Most percutaneous sacroiliac joint fixation techniques include passing a Kirschner wire (“K-wire”) or Steinmann pin through the ilium into the sacrum via a lateral approach, followed by bone site preparation and placement of one or more fixation devices. These fixation devices may include a variety of longitudinal screws and/or triangular implants that are configured to be passed across the sacroiliac joint and anchored into the soft cancellous bone of the sacrum. Thereafter, bone graft may be placed into the sacroiliac joint to promote fusion.

While current percutaneous fixation systems have alleviated pain in many patients in the immediate post-operative period, insufficient sacroiliac joint fixation may lead to a failure to obtain an effective arthrodesis and/or recurrent pain in the long term. Furthermore, patients with osteopenia or osteoporosis simply do not qualify for such procedures due to an increased risk in failure of an arthrodesis.

Therefore, there remains a need for sacroiliac joint stabilization fixation systems with improved sacral anchoring to provide better long-term compression of the joint space.

### SUMMARY OF THE INVENTION

This disclosure includes embodiments of injection devices and methods of using injection devices that comprise a conduit having an elbow configured to direct hardening material flowing in the conduit into one or more cavities.

Some embodiments of the present injection devices for use in sacroiliac joint stabilization comprise: an elongated conduit having a first end and a second end, where the first end includes a first opening and the second end includes a second opening; a carrier member couplable with the conduit and configured to accommodate a guidewire; a tool configured to be at least partially disposed in a bore of the conduit via the second opening, where the tool is configured to urge a hardening material in the bore toward the first end of the conduit; where the first opening of the conduit is configured to be angularly disposed relative to the second opening of the conduit such that the first end directs hardening material in a non-parallel direction relative to a longitudinal axis of the conduit.

In some embodiments of the present devices, the tool is longitudinally movable relative to the conduit toward the first end to urge the hardening material.

In some embodiments of the present devices, the first end of the conduit includes an elbow configured to provide the hardening material in the conduit a change in direction between the first opening and the second opening.

In some embodiments of the present devices, the elbow includes a bend angle of any one of, or between any two of: 0, 10, 15, 20, 25, 30, 35, 40, 45, 50, 55, 60, 65, 70, 75, 80, 85, 90, and 95 degrees, where the bend angle is measured between a centerline of the first opening and the longitudinal axis of the conduit.

In some embodiments of the present devices, the elbow includes an extension portion and at least a portion of the extension portion is configured to extend laterally beyond a lateral dimension of the conduit.

In some embodiments of the present devices, the lateral dimension is a radius measured from the longitudinal axis of the conduit.

In some embodiments of the present devices, the conduit and the carrier member each include a substantially circular cross-section.

In some embodiments of the present devices, the carrier member comprises a first end and a second end, the first end of the carrier member being configured to extend beyond the first end of the conduit such that, when the first opening of the conduit is disposed in a cavity, at least a portion of the first end of the carrier member is disposed outside of the cavity.

Some embodiments of the present methods (e.g., of injecting a hardening material for sacroiliac joint stabilization) comprise: inserting a guidewire across a sacroiliac joint (SI joint), such that the guidewire at least partially extends into a sacrum and an ilium; guiding an injection device toward the SI joint, where the injection device includes: an elongated conduit having a first end and a second end, where the first end includes a first opening and the second end includes a second opening; a carrier member couplable to the conduit and configured to accommodate the guidewire and thereby movably couple the conduit and the guidewire; where the first opening of the conduit is configured to be angularly disposed relative to the second opening of the conduit such that the first end directs hardening material in a non-parallel direction relative to a longitudinal axis of the conduit; injecting a first predetermined volume of hardening material into a first cavity in a cancellous bone portion of the sacrum; injecting a second predetermined volume of hardening material into a second cavity in a cancellous bone portion of the ilium; inserting a first portion of a fastener through the first cavity; and inserting a second portion of the fastener through the second cavity, thereby rigidly fixing the sacrum relative to the ilium.

Some embodiments of the present methods further comprise: forming the first cavity and second cavity using an expansion device.

In some embodiments of the present methods, the first predetermined volume of hardening material is injected using a tool configured to be at least partially disposed in a bore of the conduit via the second opening, where the tool is configured to urge the hardening material in the bore toward the first end of the conduit.

In some embodiments of the present methods, the first end of the conduit includes an elbow configured to provide the hardening material a change in direction between the first opening and the second opening.

In some embodiments of the present methods, the elbow includes a bend angle of any one of, or between any two of: 0, 10, 15, 20, 25, 30, 35, 40, 45, 50, 55, 60, 65, 70, 75, 80, 85, 90, and 95 degrees, where the bend angle is measured between a centerline of the first opening and the longitudinal axis of the conduit.

In some embodiments of the present methods, the elbow includes an extension portion and at least a portion of the extension portion is configured to extend laterally beyond a lateral dimension of the conduit.

Some embodiments of the present methods further comprise: successively disposing the elbow into the first and second cavity to inject the first and second predetermined volumes of hardening material, respectively.

Some embodiments of the present methods further comprise: injecting a respective first predetermined volume of hardening material into a plurality of cavities in the cancellous bone portion of the sacrum.

Some embodiments of the present methods further comprise: injecting a respective second predetermined volume of hardening material into a plurality of cavities in the cancellous bone portion of the ilium.

Some embodiments of the present methods further comprise: inserting a respective fastener in at least one of the plurality of cavities in the cancellous bone portion of the sacrum and at least one of the plurality of cavities in the cancellous bone portion of the ilium.

The term “coupled” is defined as connected, although not necessarily directly, and not necessarily mechanically. Two items are “couplable” if they can be coupled to each other. Unless the context explicitly requires otherwise, items that are couplable are also decouplable, and vice-versa. One non-limiting way in which a first structure is couplable to a second structure is for the first structure to be configured to be coupled (or configured to be couplable) to the second structure. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the term “substantially” may be substituted with “within [a percentage] of” what is specified, where the percentage includes 0.1, 1, 5, and 10 percent.

Further, a device or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus that “comprises,” “has,” or “includes” one or more elements possesses those one or more elements, but is not limited to possessing only those elements. Likewise, a method that “comprises,” “has,” or “includes” one or more steps possesses those one or more steps, but is not limited to possessing only those one or more steps.

Any embodiment of any of the apparatuses, systems, and methods can consist of or consist essentially of—rather than comprise/include/have—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Some details associated with the embodiments described above and others are described below.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a first cross-sectional view of one embodiment of the present injection devices, shown with some components omitted.

FIGS. 2 and 3 are the first and a second cross sectional views, respectively, of the device of FIG. 1, shown with a tool disposed therein.

FIG. 4 is an enlarged view of a portion of the device of FIG. 1.

FIGS. 5 and 6 are cross-section views of a sacroiliac joint and an expandable tool suitable for use with some embodiments of the present injection devices, wherein the expandable tool is shown in a first position and a second position, respectively.

FIGS. 7 and 8 are cross-section views of the sacroiliac joint of FIGS. 5 and 6 and the device of FIG. 1 in a first position and a second position, respectively.

FIG. 9 is a cross-section view of the sacroiliac joint of FIGS. 5 and 6, shown with a fastener.

### DESCRIPTION OF ILLUSTRATIVE EMBODIMENTS

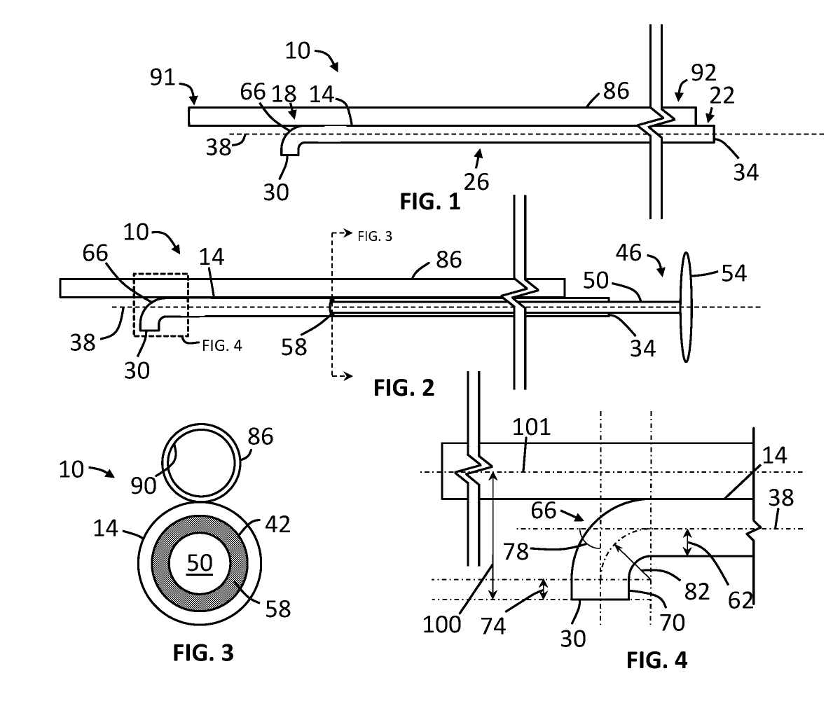

Referring now to the drawings, and more particularly to FIGS. 1-4, shown therein and designated by the reference numeral 10 is one embodiment of the present injection devices. FIGS. 1-4 are not drawn to scale. In the embodiment shown, device 10 includes an elongated conduit 14 having a first end 18, a second end 22, and a midsection 26 extending between the first end and the second end. As shown, first end 18 of conduit 14 includes a first opening 30 and second end 22 of the conduit includes a second opening 34. Conduit 14 may be characterized by and described relative to a longitudinal axis 38 extending along midsection 26.

In the embodiment shown, a bore 42 of conduit 14 may be configured to accommodate a tool 46 (e.g., a plunger, in the embodiment shown) at second opening 34 of the conduit. In this embodiment, conduit 14 can be configured to convey a hardening material (discussed in further detail below) from second end 22 towards first end 18. For example, tool 46 may be configured to urge hardening material in conduit 14 (e.g., from second end 22 of the conduit) towards first end 18 of the conduit. As shown, tool 46 may be insertable into conduit 14 and moveable relative to the conduit to urge hardening material therethrough. In the depicted embodiment, tool 46 includes a shaft 50 and one or more seals 58 (e.g., one seal, as shown), such as an o-ring, configured to sealingly engage an inner surface of conduit 14. In use, seals 58 of tool 46 may be movable (e.g., via handling of shaft 50 and/or a handle 54 that is coupled to the shaft) in bore 42 of conduit 14 along longitudinal axis 38 to urge hardening material toward first end 18 of the conduit. In some embodiments, a tool (e.g., 46) can be configured to urge hardening material through a conduit (e.g., 14) without the use of one or more seals (e.g., 58). In some embodiments, any suitable tool (e.g., 46) configured to urge hardening material in a bore (e.g., 42) of a conduit (e.g., 14) may be used, such as, for example, a pump, a shunt, one or more bone cement injectors and/or mixers, as is known in the art, and/or the like.

In the depicted embodiment, at least a portion of (e.g., the inner and/or outer surface of) conduit 14 comprises a circular cross-section. As shown, at least a portion of midsection 26 is substantially straight. More particularly, at least a portion of conduit 14 (e.g., at midsection 26) includes a substantially uniform lateral (e.g., outer) dimension 62 (e.g., a radius, as shown) measured in a direction perpendicular to longitudinal axis 38 of the midsection. Shaft 50 may include a (e.g., circular) (e.g., inner and/or outer) cross-section corresponding to the (e.g., circular) (e.g., inner and/or outer) cross-section of conduit 14. In some embodiments, a conduit (e.g., 14) and/or a shaft (e.g., 50) of a tool (e.g., 46) may comprise any appropriate uniform and/or non-uniform lateral dimension (e.g., 62) and/or any other suitable cross section, such as, for example, rectangular, square, triangular, otherwise polygonal, circular, elliptical, or otherwise round, and/or the like, such that the tool can urge hardening material in the conduit. In the embodiment shown, conduit 14 and/or tool 46 may include any suitable biocompatible material, such as plastic, steel (e.g., stainless steel), titanium, rubber, and/or the like.

In the embodiment shown, first opening 30 is configured to be angularly disposed relative to second opening 34 such that the first opening directs hardening material in a non-parallel direction relative to longitudinal axis 38. For example, in this embodiment, first end 18 of conduit 14 includes an elbow 66 configured to direct hardening material in a non-parallel direction relative to longitudinal axis 38. Elbow 66 may be unitary with conduit 14. In some embodiments, an elbow (e.g., 66) may be coupled to a conduit (e.g., 14) by, for example, one or more fasteners, a threaded connection and/or an adhesive, a weld metal, and/or any other suitable bonding material. In the embodiment shown, elbow 66 may comprise an elbow extension 70, where at least a portion of the elbow extension extends laterally beyond lateral dimension 62 of conduit 14. As shown, elbow extension 70 may be configured to have any appropriate length 74, such as, for example, any one of, or between any two of: 10, 20, 30, 40, 50, 60, 70, 80, 90, and 100 percent of lateral dimension 62. In the depicted embodiment, elbow 66 (e.g., elbow extension 70) may be configured to define first opening 30 of conduit 14 at an end of the elbow (e.g., at an end of the elbow extension), such that the first opening is disposed laterally beyond lateral dimension 62 of the conduit.

In the depicted embodiment, elbow 66 includes a bend angle 78 measured by a centerline of first opening 30 and longitudinal axis 38 of midsection 26. As shown, bend angle 78 may be for example, approximately any one of, or between approximately any two of: 1, 5, 10, 15, 20, 25, 30, 35, 40, 45, 50, 55, 60, 65, 70, 75, 80, 85, 90, and 95 degrees. As shown, bend angle 78 is approximately 90 degrees. In this embodiment, elbow includes a radius 82 configured to provide the hardening material in conduit 14 a smooth change in direction at first end 18. Radius 82 may be a percentage of lateral dimension 62 of midsection 26, such as, for example, any one of, or between any two of: 40, 80, 100, 120, 160, 200, 240, 280, 320, 360, and 400 percent of the lateral dimension. In some embodiments, an elbow (e.g., 66) may be configured to include a sharp turn such that, for example, a radius (e.g., 82) of the elbow is substantially zero. In some embodiments, an elbow extension (e.g., 70) may be omitted and a first opening 30 may be defined by an elbow (e.g., 66) along any suitable distance along a radius (e.g., 82) thereof, such that the first opening is configured to be angularly disposed relative to a second opening (e.g., 34).

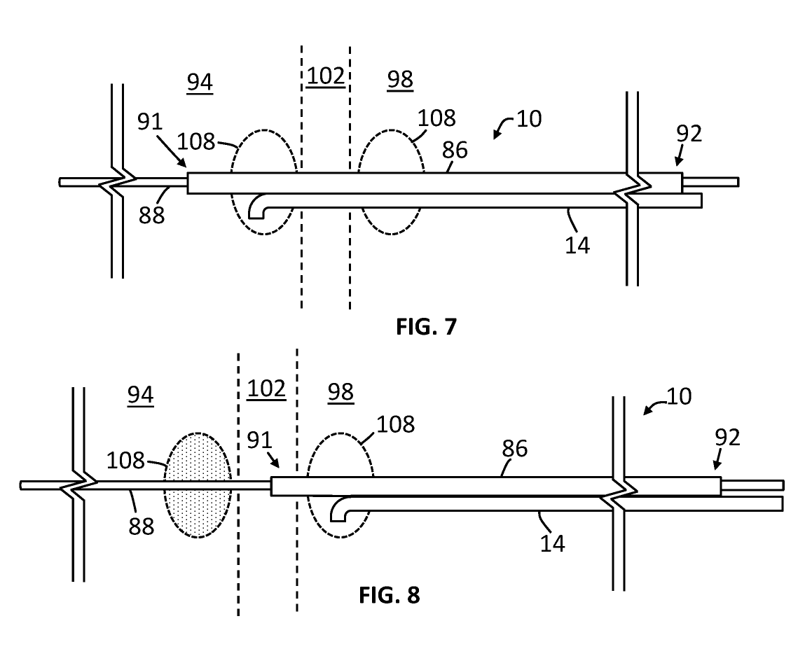

In the depicted embodiment, device 10 includes a carrier member 86 configured to movably couple conduit 14 and a guidewire 88 (e.g., such as a Kirschner wire (“K-wire”), Steinmann pin, and/or the like). In the depicted embodiment, carrier member 86 is coupled to conduit 14. As shown, carrier member 86 includes a conduit having a bore 90 configured to accommodate guidewire 88. As shown, carrier member 86 comprises a first end 91 and a second end 92, wherein the first end of the carrier member is configured to extend beyond first opening 30 of conduit 14 (e.g., which may be defined by elbow 66) such that, when the first opening is disposed in a cavity (e.g., 108), at least a portion of the first end of the carrier member is disposed outside of the cavity (e.g., see FIGS. 7 and 8). Carrier member 86 may be coupled to conduit 14 using any suitable fastener, bonding technique, and/or bonding material configured to provide sufficient strength and durability such that the carrier member and the conduit remain securely joined. In this embodiment, carrier member 86 may include any suitable biocompatible material, such as, for example, plastic, rubber, steel (e.g., stainless steel), titanium, and/or the like. In some embodiments, a carrier member (e.g., 86) may be unitary with a conduit (e.g., 14).

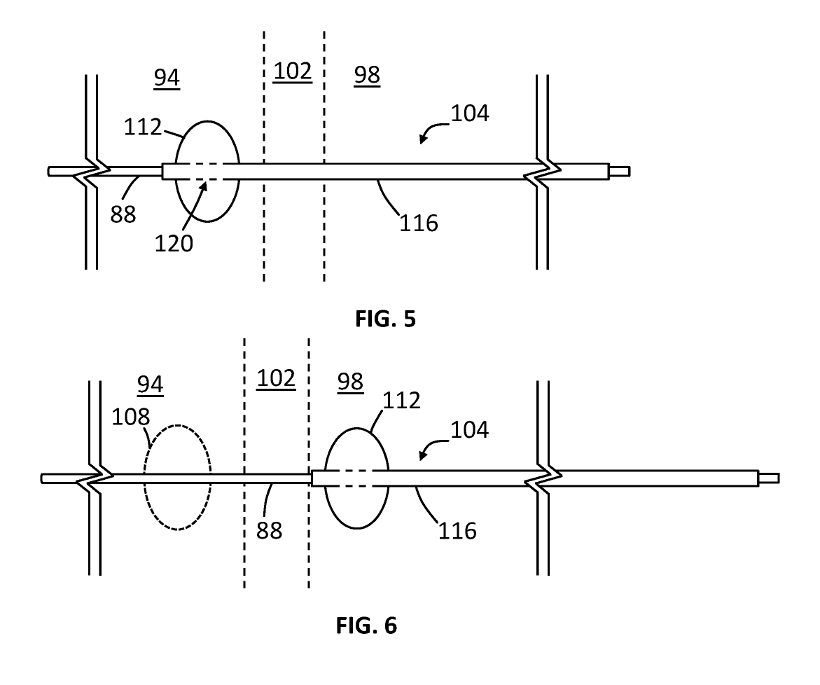

FIGS. 5-8 show a view of a sacrum 94, an ilium 98, and a sacroiliac joint 102 (“SI joint”) between the sacrum and the ilium. FIGS. 5-8 are not drawn to scale. In the depicted embodiment, guidewire 88 may be inserted in a lateral trajectory across ilium 98 and into sacrum 94. Thereafter, a space around guidewire 88 may be enlarged using drills, taps, a combination thereof, and/or the like. For example, a space around guidewire 88 may be enlarged such that device 10 can be inserted over the guidewire and into the enlarged space around the guidewire. To illustrate, a transverse dimension 100 measured, perpendicular to a longitudinal axis 101 of carrier member 86, from the longitudinal axis of the carrier member to an outermost portion of conduit 14 (e.g., elbow extension 70) may be selected such that device 10 is insertable over guidewire 88 and can occupy at least a portion of the enlarged space surrounding the guidewire. To illustrate, for example, dimension 100 may be configured to be substantially equal to or less than a radius of a device (e.g., a drill, a tap, and/or the like) used to enlarge the space around guidewire 88. In some embodiments, in preparation for bone placement, an SI joint (e.g., 102) may be at least partially cleared and/or cleaned using one or more (e.g., angled) curettes.

An expandable device 104 configured to create one or more cavity(ies) 108 in each of sacrum 94 and/or ilium 98 can be guided along guidewire 88 such that the expandable device extends into at least one of the sacrum and the ilium, as shown, for example, in FIG. 5. In this embodiment, expandable device 104 includes an expandable body 112 (e.g., a balloon) and an elongated conduit 116 on which expandable body 112 is disposed. As shown, conduit 116 may be configured to accommodate guidewire 88 and/or deliver hardening material to expandable body 112 via one or more aperture(s) 120 in conduit 116. Exemplary examples of expandable bodies (e.g., 112) used with sacroiliac joints are disclosed in U.S. Pat. No. 8,551,171 entitled “Methods of stabilizing the sacroiliac joint,” which is hereby incorporated by reference in its entirety.

In the depicted embodiment, expandable body 112 may be inserted into a patient while expandable body 112 is in a contracted position. After expandable device 104 and expandable body 112 are appropriately positioned in the patient, such as, for example, in the cancellous bone of the sacrum and/or the ilium, the expandable body can be expanded to an appropriate size (e.g., an expanded position), as shown in FIG. 5. For example, in this embodiment, conduit 116 may be in fluid communication with an expansion pump configured to inflate expandable body 112 using a fluid (e.g., a contrast media). In this embodiment, the expansion pump may measure an applied pressure and an amount of fluid delivered into expandable device 104 and/or expandable body 112, thereby controlling the size of the expandable body 112 in the expanded position.

As shown, expansion of expandable body 112 in sacrum 94 can create one or more cavities 108 in the cancellous bone of the sacrum. Similarly, expansion of expandable body 112 in ilium 98 can create one or more cavities 108 in the cancellous bone of the ilium. For example, expandable body 112 may be contracted and/or expanded (e.g., using the expansion pump) and expandable device 104 may be moved across SI joint 102 such that the expandable body is appropriately positioned in the cancellous bone of ilium 98 and/or the cancellous bone of sacrum 94 to create cavity(ies) 108. In some embodiments, an expandable device (e.g., 104) includes a plurality of expandable bodies (e.g., 112) longitudinally spaced on the expansion device and configured to form respective cavities (e.g., 108) on opposing sides of an SI joint (e.g., 102) simultaneously and/or sequentially (e.g., while keeping the expansion device in a single position). In some embodiments, one or more cavities (e.g., 108) may be created on only a single side of an SI joint (e.g., 102), such as, for example, only within the cancellous bone of a sacrum (e.g., 94) or only within the cancellous bone of an ilium (e.g., 98).

In this embodiment, after forming cavity(ies) 108 in sacrum 94 and/or ilium 98, expandable device 104 may be removed from the patient. Thereafter, device 10 is guided via guidewire 88 towards cavity(ies) 108 in sacrum 94 and/or ilium 98. In the depicted embodiment, carrier member 86 is fitted over guidewire 88 (e.g., the guidewire is positioned in bore 90 of the carrier member) such that conduit 14 is guided toward cavity(ies) 108 in sacrum 94 and/or ilium 98.

As shown, for example, in FIG. 7, device 10 may be moved into a first position, where the device traverses SI joint 102, and first opening 30 of conduit 14 is disposed in cavity 108 of sacrum 94. Thereafter, a hardening material may be provided into conduit 14 and tool 46 may be inserted in bore 42 of the conduit behind the hardening material. For example, such a hardening material can comprise bone cement, such as, for example, poly(methyl methacrylate) (PMMA) and/or the like. In some embodiments, hardening material (e.g., for injection into one or more cavities (e.g., 108)) may be disposed in a conduit (e.g., 14) proximate to an opening (e.g., 30) at the time an injection device (e.g., 10) is inserted into a patient such that the amount of air injected into the cavity(ies) is reduced. In some embodiments, an additional volume of hardening material may be injected into a conduit (e.g., 14) after an injection device (e.g., 10) is inserted into a patient.

In this embodiment, tool 46 may be moved towards first end 18 of conduit 14 (e.g., by a first predetermined distance along longitudinal axis 38), thereby urging the hardening material through the conduit and injecting a first predetermined volume of the hardening material into cavity 108 in sacrum 94. For example, by urging tool 46 towards first end 18 of conduit 14, hardening material is directed out of first opening 30 of the conduit via elbow 66. The first predetermined volume of hardening material injected into cavity 108 of sacrum 94 may be substantially equal to or greater than a volume of the cavity of the sacrum. In some embodiments, a first predetermined volume of hardening material injected into a cavity (e.g., 108) of a sacrum (e.g., 94) may be less than a volume of the cavity of the sacrum. In this embodiment, after the first predetermined volume of hardening material has been injected into cavity 108 of sacrum 94, injection of the hardening material is suspended, such as, for example, by stopping the movement of tool 46 relative to conduit 14.

Device 10 may be moved into a second position, where first opening 30 of conduit 14 is disposed in cavity 108 of ilium 98. In this embodiment, tool 46 may be moved towards first end 18 of conduit 14 (e.g., by a second predetermined distance along longitudinal axis 38), thereby urging the hardening material through the conduit and injecting a second predetermined volume of the hardening material into cavity 108 in ilium 98. For example, by urging tool 46 towards first end 18 of conduit 14, the hardening material is directed out of first opening 30 of the conduit via elbow 66. The second predetermined volume of hardening material injected into cavity 108 of ilium 98 may be substantially equal to or greater than a volume of the cavity of the ilium. In some embodiments, a second predetermined volume of hardening material injected into a cavity (e.g., 108) of an ilium (e.g., 98) may be less than a volume of the cavity of the ilium. In this embodiment, after the second predetermined volume of hardening material has been injected into cavity 108 of ilium 98, injection of the hardening material is suspended, such as, for example, by stopping the movement of tool 46 relative to conduit 14. In embodiments where one or more cavities (e.g., 108) are created on opposing sides of an SI joint (e.g., 102), a device (e.g., 10) can inject hardening material into the cavity(ies) of a sacrum (e.g., 94) and cavity(ies) of an ilium (e.g., 98) in any suitable order, such as, for example, first into cavity(ies) of the sacrum and then into cavity(ies) of the ilium or first into cavity(ies) of the ilium and then into cavity(ies) of the sacrum. In embodiments where cavity(ies) (e.g., 108) are created only on a single side of an SI joint (e.g., 102) such as, for example, only within the cancellous bone of a sacrum (e.g., 94) or only within a cancellous bone of an ilium (e.g., 98), a device (e.g., 10) can inject hardening material into the cavity(ies) on that particular side of the SI joint. Thereafter, device 10 may be removed from the patient.

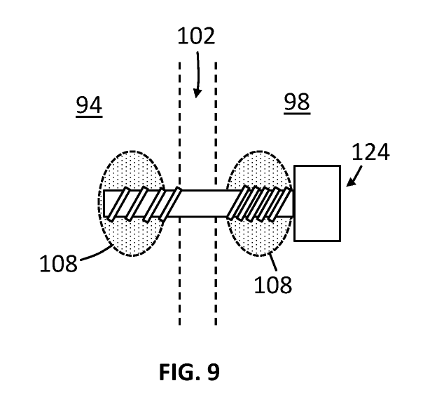

Referring now to FIG. 9 (not drawn to scale), in this embodiment, one or more fasteners 124 can be configured to be disposed at least partially within cavity(ies) 108 of sacrum 94 and/or cavity(ies) 108 of ilium 98. For example, in the embodiment shown, hardening material has been injected into cavity(ies) 108 of sacrum 94 and cavity(ies) 108 of ilium 98 and device 10 has been removed from the patient. Fastener(s) 124 (e.g., one fastener, as shown), such as, for example, a sacroiliac joint screw, can be configured to be guided (e.g., by guidewire 88) across SI joint 102 such that the fastener(s) engage hardening material in cavity(ies) 108 in sacrum 94 and cavity(ies) 108 ilium 98. In embodiments where cavity(ies) (e.g., 108) are created only on a single side of an SI joint (e.g., 102) such as, for example, only within the cancellous bone of a sacrum (e.g., 94) or only within a cancellous bone of an ilium (e.g., 98), fastener(s) (e.g., 124) can be configured such that the fastener(s) are disposed at least partially within the cavity(ies) on that particular side of the SI joint and the fastener(s) at least partially extend across the SI joint. In some embodiments, a guidewire (e.g., 88) may be removed from a sacrum (e.g., 94) and/or an ilium (e.g., 98) before fastener(s) (e.g., 124) are inserted across an SI joint (e.g., 102) to, for example, prevent adhesion between the guidewire and hardening material in one or more cavities (e.g., 108).

Fastener(s) 124 are configured such that, after disposing the fastener(s) within one or more cavity(ies) 108 and at least partially across SI joint 102, the fastener(s) prevent movement of cavity(ies) 108 in sacrum 94 and/or ilium 98 relative to the SI joint, thereby preventing relative movement between the sacrum and the ilium.

Fastener(s) (e.g., 124) can comprise any suitable biocompatible material, such as plastic, steel (e.g., stainless steel), titanium, rubber, and/or the like. Fastener(s) (e.g., 124) may include any suitable type of implant, such as, for example an implant having a cross-sectional shape that is rectangular, square, triangular, otherwise polygonal, circular, elliptical, or otherwise round, a screw (e.g., a dual-threaded screw, an elongated screw, a cannulated screw, a fenestration screw, and/or the like screws), and/or the like. In some embodiments, fastener(s) (e.g., 124) having dual threads may be of an unequal pitch such that a distal portion thereof advances at a rate greater than a proximal portion. Moreover, in such embodiments, the dual threads may be configured to pull a sacral (e.g., 94) bone and an iliac (e.g., 98) bone together as the fastener(s) (e.g., 124) are advanced across an SI joint (e.g., 102) via a guidewire (e.g., 88). In these embodiments, the threading of the proximal portion may be configured to allow the fastener(s) to rigidly affix to the lateral cortical wall. In some embodiments, one or more fastener(s) (e.g., 124) may include one or more fenestration(s) disposed in a proximal portion and/or a distal portion such that one or more fenestration(s) are disposed on each side of an SI joint (e.g., 102). In some embodiments, one or more fastener(s) (e.g., 124) may be configured to promote fusion of an SI joint (e.g., 102) by providing bone craft into the SI joint.

The above-described embodiments include the benefits of providing strong constructs that can sustain the weight of the patient's body, resist rotational and/or translational forces across the SI joint, promote sacroiliac joint fusion over time, and/or take advantage of the sacral cortical layer to resist pull-out.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An injection device for use in sacroiliac joint stabilization, comprising:
an elongated conduit having a first end and a second end, where the first end includes a first opening and the second end includes a second opening;
a carrier member couplable with the conduit and configured to accommodate a guidewire;
a tool configured to be at least partially disposed in a bore of the conduit via the second opening, where the tool is configured to urge a hardening material in the bore toward the first end of the conduit;
where the first opening of the conduit is configured to be angularly disposed relative to the second opening of the conduit such that the first end directs the hardening material in a non-parallel direction relative to a longitudinal axis of the conduit.

2. The device of claim 1, where the tool is longitudinally movable relative to the conduit toward the first end to urge the hardening material.

3. The device of claim 1, where the first end of the conduit includes an elbow configured to provide the hardening material in the conduit a change in direction between the first opening and the second opening.

4. The device of claim 3, where the elbow includes a bend angle of any one of, or between any two of: 0, 10, 15, 20, 25, 30, 35, 40, 45, 50, 55, 60, 65, 70, 75, 80, 85, 90, and 95 degrees, where the bend angle is measured between a centerline of the first opening and the longitudinal axis of the conduit.

5. The device of claim 3, where the elbow includes an extension portion and at least a portion of the extension portion is configured to extend laterally beyond a lateral dimension of the conduit.

6. The device of claim 5, where the lateral dimension is a radius measured from the longitudinal axis of the conduit.

7. The device of claim 1, where the conduit and the carrier member each include a substantially circular cross-section.

8. The device of claim 1, where the carrier member comprises a first end and a second end, the first end of the carrier member being configured to extend beyond the first end of the conduit such that, when the first opening of the conduit is disposed in a cavity, at least a portion of the first end of the carrier member is disposed outside of the cavity.

9. A method of injecting a hardening material for sacroiliac joint stabilization, comprising:
inserting a guidewire across a sacroiliac joint (SI joint), such that the guidewire at least partially extends into a sacrum and an ilium;
guiding an injection device toward the SI joint, where the injection device includes:
an elongated conduit having a first end and a second end, where the first end includes a first opening and the second end includes a second opening;
a carrier member couplable to the conduit and configured to accommodate the guidewire and thereby movably couple the conduit and the guidewire;
where the first opening of the conduit is configured to be angularly disposed relative to the second opening of the conduit such that the first end directs hardening material in a non-parallel direction relative to a longitudinal axis of the conduit;
injecting a first predetermined volume of hardening material into a first cavity in a cancellous bone portion of the sacrum;
injecting a second predetermined volume of hardening material into a second cavity in a cancellous bone portion of the ilium;
inserting a first portion of a fastener through the first cavity; and
inserting a second portion of the fastener through the second cavity, thereby rigidly fixing the sacrum relative to the ilium.

10. The method of claim 9, further comprising forming the first cavity and second cavity using an expansion device.

11. The method of claim 9, where the first predetermined volume of hardening material is injected using a tool configured to be at least partially disposed in a bore of the conduit via the second opening, where the tool is configured to urge the hardening material in the bore toward the first end of the conduit.

12. The methods of claim 9, where the first end of the conduit includes an elbow configured to provide the hardening material a change in direction between the first opening and the second opening.

13. The method of claim 12, where the elbow includes a bend angle of any one of, or between any two of: 0, 10, 15, 20, 25, 30, 35, 40, 45, 50, 55, 60, 65, 70, 75, 80, 85, 90, and 95 degrees, where the bend angle is measured between a centerline of the first opening and the longitudinal axis of the conduit.

14. The method of claim 12, where the elbow includes an extension portion and at least a portion of the extension portion is configured to extend laterally beyond a lateral dimension of the conduit.

15. The method of claim 12, further comprising successively disposing the elbow into the first and second cavity to inject the first and second predetermined volumes of hardening material, respectively.

16. The method of claim 9, further comprising injecting a respective first predetermined volume of hardening material into a plurality of cavities in the cancellous bone portion of the sacrum.

17. The method of claim 16, further comprising injecting a respective second predetermined volume of hardening material into a plurality of cavities in the cancellous bone portion of the ilium.

18. The method of claim 17, further comprising inserting a respective fastener in at least one of the plurality of cavities in the cancellous bone portion of the sacrum and at least one of the plurality of cavities in the cancellous bone portion of the ilium.
